(19) United States
(12) Patent Application Publication
(10) Pub. No.: US 2022/0054311 A1
(43) Pub. Date: Feb. 24, 2022

(54) Eye Drop Dispensing Apparatus

(71) Applicant: United States Government As Represented By The Department of Veterans Affairs, Washington, DC (US)

(72) Inventor: Terri Ohlinger, Fairfield, OH (US)

(21) Appl. No.: 17/399,623

(22) Filed: Aug. 11, 2021

Related U.S. Application Data
(60) Provisional application No. 63/064,536, filed on Aug. 12, 2020.

(51) Int. Cl. A61F 9/00

(52) U.S. Cl. CPC A61F 9/0026

(57) ABSTRACT

An apparatus for dispensing eye drops can comprise a housing that defines a receptacle. The receptacle can be configured to receive a cartridge having a solution therein. An actuator can be movable between a first position and a second position. The actuator can be positioned with respect to the receptacle so that, when the cartridge is received in the receptacle and the actuator is moved from the first position to the second position, the actuator deforms the cartridge by a select amount to dispense a predetermined quantity of solution from the cartridge.

### CROSS-REFERENCE TO RELATED PATENT APPLICATION

This application claims priority to and the benefit of the filing date of U.S. Provisional Patent Application Ser. No. 63/064,536, filed Aug. 12, 2020, which is incorporated herein by reference in its entirety.

### FIELD

0 This application relates to apparatuses that assist users with dispensing eye drops.

### BACKGROUND

Self-administering eye drops into one's eye(s) can he a difficult task that requires a steady hand to aim an. eye dropper and refined motor skills to squeeze the eye dropper to dispense a select amount of solution (e.g., one or two drops). Accordingly, individuals having, tremors or reduced mobility have trouble aligning the eye dropper above the eye. Further, those who have weak hand strength or control over hand movements (e.g., such as individuals with Parkinson's disease or arthritis) have difficulty applying a select amount of pressure to dispense the drops from the dropper. Accordingly, a device that assists the individual with dropper alignment and controlled dispensing of drops from the dropper.

### SUMMARY

Disclosed herein, in one aspect, is an apparatus for dispensing eye drops. The apparatus can comprise a housing that defines a receptacle. The receptacle can be configured to receive a cartridge having a solution therein. An actuator can be movable between a first position and a second position. The actuator can he positioned with respect to the receptacle so that, when the cartridge is received in the receptacle and the actuator is moved from the first position to the second position, the actuator deforms the cartridge by a select amount to dispense a predetermined quantity of solution from the cartridge.

In another aspect, an apparatus can comprise a housing that defines at least a first receptacle that is configured to receive and support a cartridge having a solution therein. The first receptacle can have a central axis. The apparatus can include an actuator that is coupled to the housing and movable between a first position and a second position. The actuator can comprise an engagement element. In the first position, the engagement element of the actuator can be radially spaced from the central axis of the first receptacle by a first distance, and in the second position, the engagement element of the actuator can be positioned within the first receptacle and radially spaced from the central axis of the first receptacle by a second distance that is less than the first distance. Optionally, the housing can define an alignment feature that is at least partially receivable into an eye socket of a user so that when the housing is positioned above an eye of the user and the alignment feature is received within the eye socket of the user, the alignment feature is configured to align the central axis of the first receptacle with the eye of the user.

In another aspect, an apparatus for use with a user having a face, a first eye, and a second eye can comprise a frame that defines at least one alignment feature. The at least one alignment feature can be configured to engage the face of the user to align the frame in a predetermined orientation with respect to the face of the user. A first receptacle can be configured to align a cartridge containing solution therein with respect to the first eye of the user. A second receptacle can be configured to align the cartridge containing solution therein with the second eye of the user. An actuator can be coupled to the frame and can be configured to receive the cartridge containing solution therein. The actuator can be movable between a first position and a second position, wherein, when the actuator is moved from the first position to the second position, the actuator deforms the cartridge by a select amount to dispense a predetermined quantity of solution from the cartridge.

In a further aspect, a method comprises positioning a cartridge within a first receptacle of a housing of an apparatus, the cartridge containing a solution, the first receptacle having a central axis, the apparatus further comprising an actuator that is coupled to the housing and movable between a first position and a second position, wherein the actuator comprises an engagement element, wherein in the first position, the engagement element of the actuator is radially spaced from the central axis of the first receptacle by a first distance. The method can further comprise moving the actuator from the first position to the second position, wherein in the second position, the engagement element of the actuator is positioned within the first receptacle and radially spaced from the central axis of the first receptacle by a second distance that is less than the first distance. In the second position, the engagement element of the actuator compresses the cartridge to dispense a quantity of the solution within the cartridge.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF IRE DRAWINGS

FIG. 1. is a perspective view of an eye drop dispensing apparatus in accordance with the present disclosure.

FIGS. 2A-2D are perspective views of alternative embodiments of eye drop dispensing apparatuses in accordance with embodiments of the present disclosure.

FIG. 3 is a perspective view of a wearable eye drop dispensing apparatus in accordance with embodiments disclosed herein.

FIG. 4 is a perspective view of another wearable eye drop dispensing apparatus.

FIG. 5 is a perspective view of another wearable eye drop dispensing apparatus.

FIG. 6 is a schematic of a solenoid actuator for use with any of the eye drop dispensing apparatuses as disclosed herein.

FIG. 7 is a schematic of a bladder actuator for use with any of the eye drop dispensing apparatuses as disclosed herein.

FIG. 8A is a top view of an eye drop dispensing apparatus in accordance with embodiments disclosed herein. FIG. 8B is a side view of the eye drop dispensing apparatus of FIG. 8A. FIG. 8C is a front view of the eye drop dispensing apparatus of FIG. 8A. FIG. 8D is a rear perspective view of the eye drop dispensing apparatus of FIG. 8A.

FIG. 9A is a side view of a first side of the housing of the eye drop dispensing apparatus of FIG. 8A with optional exemplary dimensions in. inches. FIG. 9B is a front view of the first side of the housing of FIG. 9A. FIG. 9C is a rear perspective view of the first side of the housing of FIG. 9A.

FIG. 10A is a side view of a second side of the housing of the eye drop dispensing apparatus of FIG. 8A with optional exemplary dimensions in inches. FIG. 10B is a front view of the second side of the housing of FIG. 10A. FIG. 10C is a rear perspective view of the second side of the housing of FIG. 10A.

FIG. 11 is a perspective view of a rack of the eye drop dispensing apparatus of FIG. 8A.

FIG. 12 is a perspective view of the pinion of the eye drop dispensing apparatus of FIG. 8A.

### DETAILED DESCRIPTION

The disclosed system and method may he understood more readily by reference to the following detailed description of particular embodiments and the examples included therein and to the Figures and their previous and following description.

It is to be understood that the terminology used herein is for the purpose of describing particular embodiments only and is not intended to limit the scope of the present invention which will be limited only by the appended claims.

It must be noted that as used herein and in the appended claims, the singular forms “a,” “an,” and “the” include plural references unless the context clearly dictates otherwise. Thus, for example, reference to “an alignment feature” or “an engagement element” includes one or more of such alignment features or engagement elements, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. it will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects when values are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is riot intended to exclude, for example, other additives, components, integers or steps that are not listed in the step.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds to any possible non-express basis for interpretation, including: matters of logic with respect to arrangement of steps or operational flow; plain meaning derived from grammatical organization or punctuation; and the number or type of aspects described in the specification. Thus, words denoting order, such as “first” or “next,” should be interpreted as optional aspects unless plain meaning or logic dictates. otherwise.

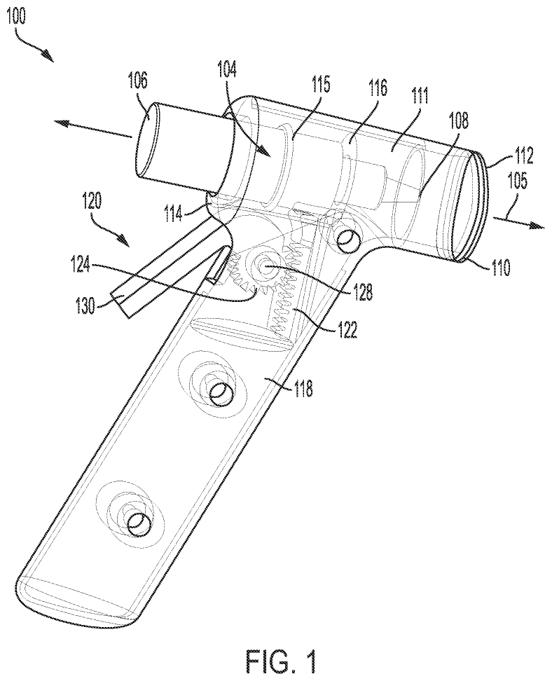

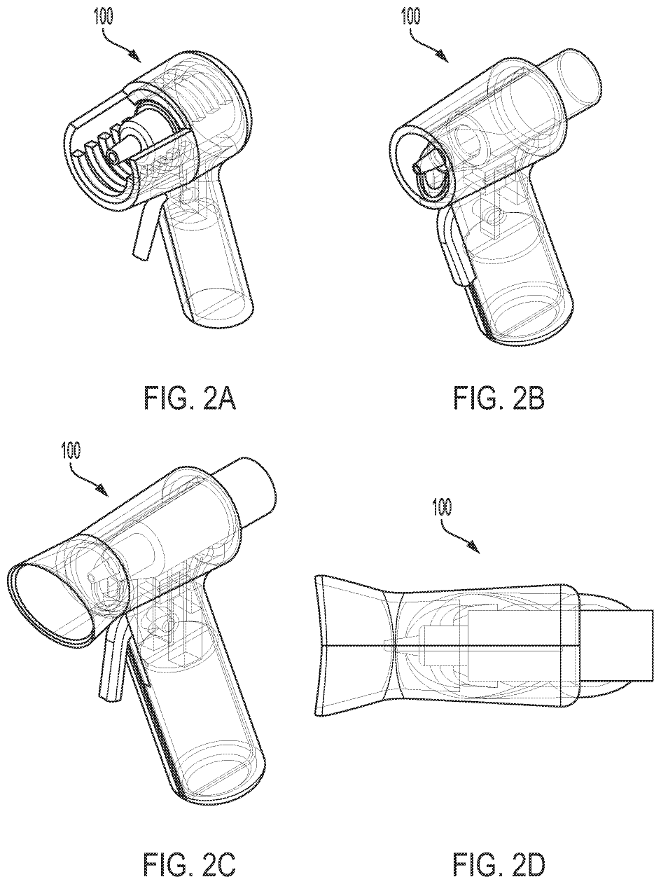

Disclosed herein, in various aspects and with reference to FIGS. 1-2D, is an eye drop dispensing apparatus 100. The eye drop dispensing apparatus 100 can comprise a housing 102 that can define a receptacle 104 for receiving a container (e.g., cartridge 106). The receptacle 104 can have a central axis 105.

The container (e.g., cartridge 106) can contain an eye drop solution (e.g., lubricating eye drops, a tear-replacing solution, or a solution containing ophthalmic drugs), and can define an outlet 108 for dispensing the eye drop solution therefrom In some optional aspects, the cartridge 106 can be a conventional eye drop container for conventional dispensing of eye drops. The cartridge can have an outlet (e.g., a nozzle) that is coaxial with, or otherwise aligned with, the central axis 105 of the receptacle 104.

The housing 102 can define an alignment feature 110 (e.g., an alignment surface). Optionally, the alignment feature 110 (e.g., alignment surface) can have dimensions for receipt into (at least partially within) an eye socket of a user. In this way, the alignment feature 110 (e.g., alignment surface) can be (at least partially) receivable into the eye socket of the user so that when housing 102 is positioned above an eye of the user and the alignment feature is received within the eye socket of the user, the alignment feature is configured to align the central axis of the first receptacle with the eye of the user, with the outlet of the cartridge 106 aligned with the eye of the user. The alignment feature 110 can optionally be a rim of an end of the housing 102 proximate the outlet of the cartridge 106 when the cartridge is received within the receptacle 104 of the housing. Optionally, the alignment feature 110 (e.g., alignment surface) can comprise a padded material to protect the skin of the user. Optionally, the alignment feature 110 can be cylindrical or generally cylindrical or frustoconical or generally frustoconical. In further aspects, the alignment feature 110 alignment surface) can be oblong, having a major dimension and a minor dimension, wherein the minor dimension is selected for receipt between the upper and lower portions of the eye socket. In further optional aspects, the alignment feature 110 (e.g., alignment surface) can be configured to bias against one or more of the user's nose, forehead, cheek, ear, or other portion of the face or head.

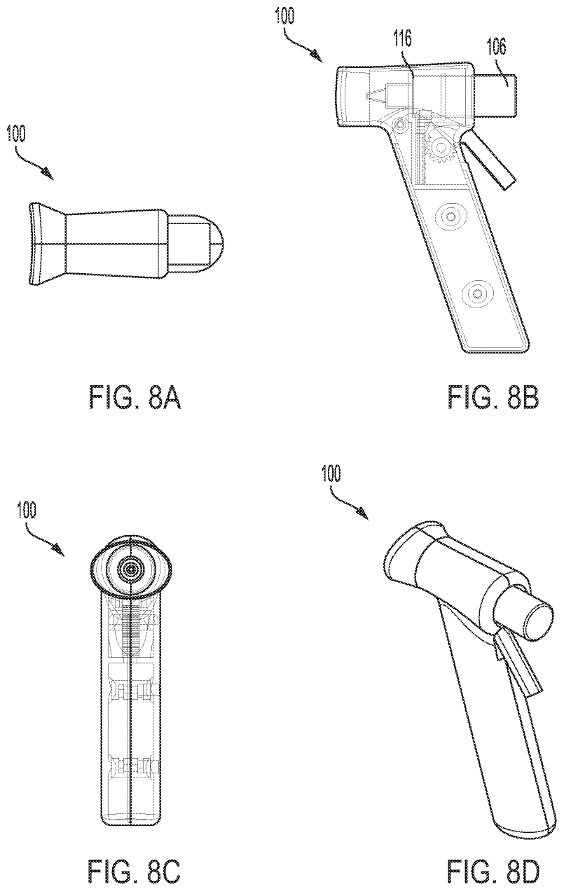

The housing 102 can optionally have a main body 111 having a first end 112 that defines the alignment feature 110 (e.g., alignment surface) and an opposing second end 114 that defines an opening to the receptacle 104. The first end 112 can define an opening to allow drops from the outlet 108 of the cartridge 106 to fall therethrough. The housing 102 can define a stop 116 (FIG. 8B) that engages the cartridge 106 when the cartridge is fully inserted in the receptacle 104. The housing 102 can further comprise an O-ring 115 for biasing against the circumferential outer surface of the cartridge 106 to retain the cartridge in the receptacle 104.

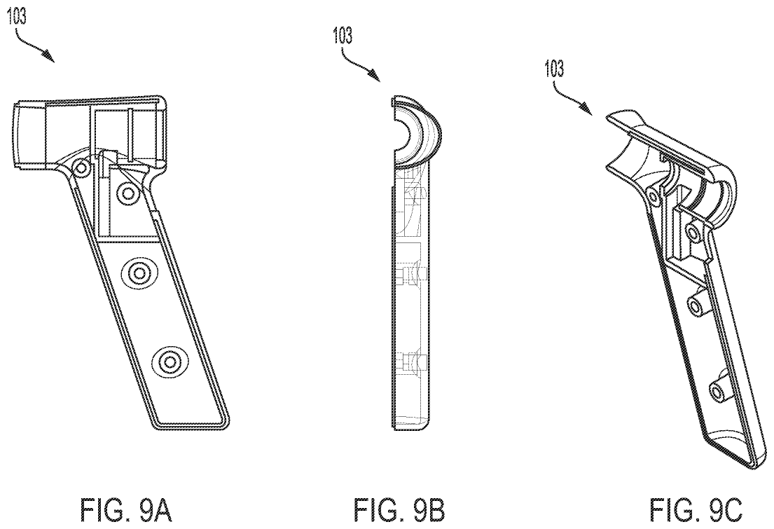

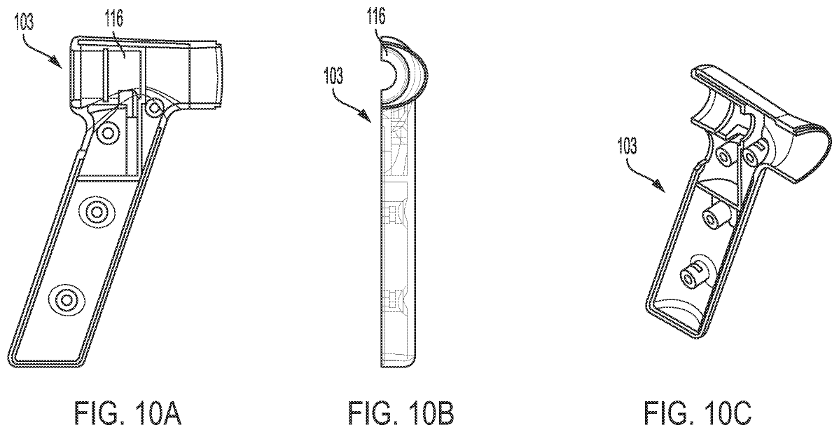

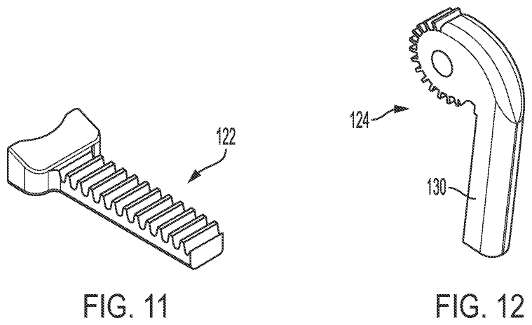

The housing 102 can comprise a handle 118. Optionally, the handle 118 can extend from the main body 111 toward a distal end 119 at an oblique angle with respect to the main body 111 and away from the first end 112. Optionally, the housing 102 can comprise two portions that are coupled via one or more fasteners. For example, the housing 102 can be formed from two opposing sections 103 (FIG. 9A-10C) that each define half, or about half, of the housing. Alternatively, the housing 102 can comprise a single, monolithic structure.

The eye drop dispensing apparatus 100 can comprise an actuator 120 that can be configured to dispense a predetermined quantity of solution from the cartridge 106. For example, the actuator 120 can be movable between a first position and a second position. The actuator 120 can be positioned with respect to the receptacle 104 so that, when the cartridge 106 is received in the receptacle and the actuator is moved from the first position to the second position, the actuator deforns the cartridge 106 by a select amount to dispense the predetermined quantity of solution from the cartridge. It is contemplated that different cartridges can dispense solution at different threshold compression threes. For example, the threshold compression forces can be between about 17.8 N and 160 N. Accordingly, the actuator 120 can facilitate controlled dispensing, particularly for individuals lacking grip strength or control. In further aspects, the actuator need not move between select first and second positions. For example, the user can stop actuating the actuator 120 (e.g., squeezing a lever) once sufficient drops have been dispensed.

In some aspects, the actuator 120 can have an engagement element. The engagement element can be radially spaced from the central axis 105 of the first receptacle 104 by a first distance when the actuator is in the first position. The engagement element can be radially spaced from the central axis 105 of the first receptacle 104 by a second distance that is less than the first distance when the actuator is in the second position.

In some aspects, the actuator 120 can comprise a rack 122 and a pinion 124. The rack 122 can serve as the engagement element of the actuator 120, wherein the rack is configured to move about and between the first distance and the second distance from the central axis 105 of the receptacle 104 (when the actuator is moved about and between the first and second positions, respectively) to compress the cartridge 106. The pinion 124 can have a rotational axis 128. The pinion 124 can comprise a lever 130 that extends radially outwardly from the rotational axis. Optionally, the eye drop dispensing apparatus IOU can comprise a stop that is configured to inhibit the actuator from moving beyond the second position, thereby providing a select volume of dispensed solution. For example, the handle 118 of the housing 108 can act as a stop that inhibits further rotation of the pinion 124, which can correspond to a select linear travel of the rack 122 that results in the dispensing of the predetermined quantity of solution. Optionally, the actuator 120 can provide a mechanical advantage (e.g., via the lever 130) so that a user with insufficient hand strength to compress the cartridge can, via the mechanical advantage, cause the cartridge to dispense drops.

The eye drop dispensing apparatus 100 can be sterile and/or configured for sterilization. Accordingly, it is contemplated that the eye drop dispensing apparatus 100 can be configured for multiple uses (optionally, by different users). The housing 102 of the eye drop apparatus 100 can comprise a material that is safe for skin contact and inhibits microorganism growth. Optionally, the eye drop dispensing apparatus 100 can comprise one or more polymer materials and can, in some aspects, be formed via additive manufacturing or via injection molding.

To use the eye drop dispensing apparatus 100, a user or caretaker can insert a cartridge 106 into the receptacle 104 of the housing. The user or a caretaker can align the apparatus 100 so that the outlet 108 of the cartridge 106 is directly above the user's eye. For example, the alignment feature 110 (e.g., alignment surface) can be received within the eye socket of the user with the user's head tilted back. The user or caretaker can actuate the actuator 120 to dispense the predetermined quantity of solution from the apparatus.

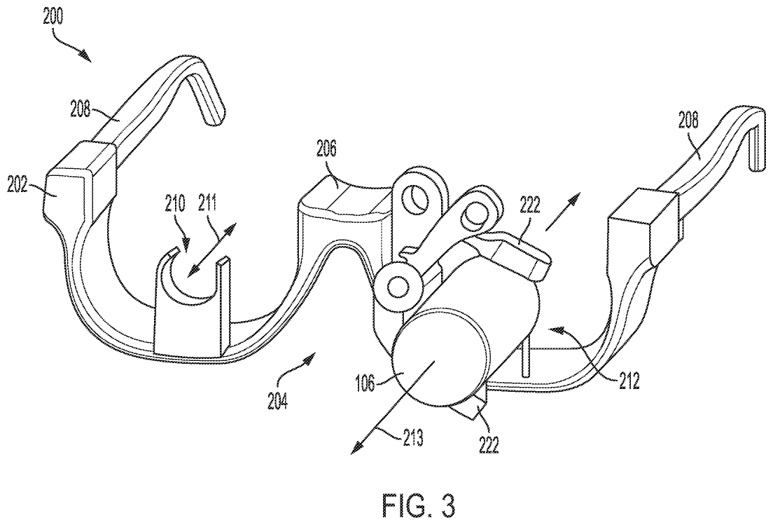

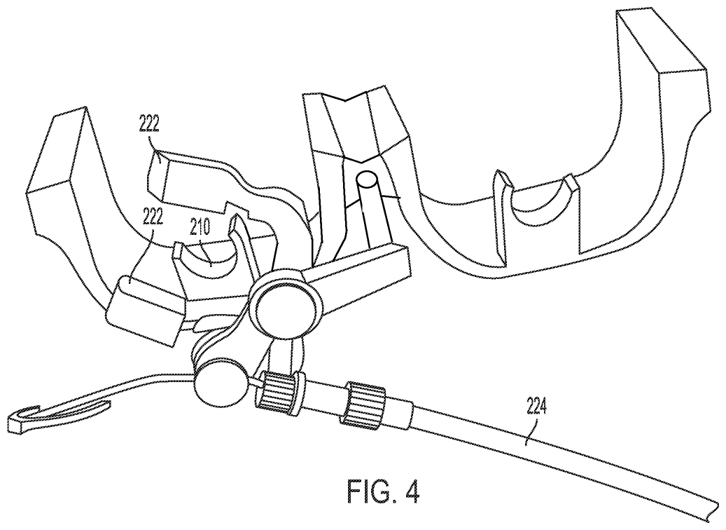

Referring to FIGS. 3-5, in another optional. embodiment, an eye drop dispensing apparatus 200 can he worn on the user's head or face. The eye drop dispensing apparatus 200 can comprise a frame 202. The frame 202 can be configured for engagement with a user's face in a predetermined alignment. For example, the frame can define one or more alignment features that engage the user's face to guide the frame 202 to the predetermined alignment. The alignment features can optionally include, for example and without limitation, a nose cutout 204 that is configured to receive the nose of the user and a forehead bridge 206 that is configured to bias against the forehead of the user, and eyeglass temples 208 that can optionally be hingedly connected to the remainder of the frame and can extend over respective ears of the user. Accordingly, the frame 202 can optionally be worn in the same way as conventional eyeglasses. In further optional aspects, the alignment features can include a chin strap (not shown) or a strap or cap (not shown) that is configured to extend over the top of the user's head. Thus, in further optional aspects, the eye drop dispensing apparatus 200 can be worn as a helmet or other head covering.

The frame 202 can define a first receptacle 210 and a second receptacle 212 that are each configured to at least partially receive a cartridge 106 containing solution therein. The first receptacle can have a central axis 211, and the second receptacle can have a central axis 213. The first receptacle 210 can be positioned so that when the frame 202 is worn on the user's face and a cartridge 106 is received within the first receptacle, the cartridge is aligned with the user's first eye for dispensing the solution into said first eye. Likewise, the second receptacle 212 can be positioned so that when the frame 202 is worn on the user's face and a cartridge 106 is received within the second receptacle, the cartridge is aligned with the user's second eye for dispensing the solution into said second eye.

The eye drop dispensing apparatus 200 can further comprise an actuator 220 that is coupled to the frame 202. The actuator 220 can be configured to receive a cartridge 106 containing solution therein. The actuator 220 is movable between a first position and a second position. When the actuator 220 is moved from the first position to the second position, the actuator deforms the cartridge 106 by a select amount to dispense a predetermined quantity of solution from the cartridge.

Optionally, the actuator 220 can comprise a pair of calipers 222 and an actuation cable 224. Each of the calipers 222 can be an engagement element of the actuator 120. With the actuator 120 in the first position, each of the calipers 222 can be spaced from the central axis 211 of the first receptacle 210 (or central axis 213 of the second receptacle 212, depending on which receptacle the calipers 222 are aligned with) by a respective first distance. With the actuator 120 in the second position, each of the calipers 222 can be spaced from the central axis 211 of the first receptacle 210 (or central axis 213 of the second receptacle 212, depending on which receptacle the calipers 222 are aligned with) by a respective second distance that is less than the respective first distance of the caliper 222. In some aspects, the calipers 222 can be pivotably coupled to each other so that retraction of the actuation cable 224 can close the calipers by a select amount. The closing of the calipers 222 can compress the cartridge 106 to dispense the predetermined quantity of solution from the cartridge. In some aspects, the actuator 220 can further comprise a handle (not shown) and a lever (not shown) that is pivotably coupled thereto so that squeezing the lever toward the handle retracts the actuation cable. in some optional aspects, the lever can be movable only by a predetermined angle with respect to the handle to limit the travel of the calipers. For example, in some aspects, the lever can pivot until it biases against the handle. Accordingly, the handle can serve as a stop that limits the pivotal angle of the lever. In some aspects, the calipers 222 can be configured to close only to a predetermined closed position (e.g., until the calipers engage a stop). Optionally, the calipers 222 can comprise gripping elements (e.g., rubber gripping elements) that engage an outer surface of the cartridge.

Optionally, the actuator 220 can be embodied as, or similar to, a bicycle brake caliper.

Optionally, the eye drop dispensing apparatus 200 can be actuated without requiring the user to move her arms above her head. Accordingly, the eye drop dispensing apparatus 200 can be particularly beneficial for those with limited mobility and/or those who have difficulty reaching above their heads.

The eye drop dispensing apparatus 200 can further comprise a swing arm 230 that is pivotably coupled to the frame 202. The swing arm 230 can be configured to receive the cartridge 106. The swing arm 230 can be moved from a first rotational position to a second rotational position. When the swing arm 230 is in the first rotational position, the cartridge 106 can be positioned within the first receptacle 210, and when the swing arm is in the second rotational position, the cartridge can be positioned within the second receptacle 212. Accordingly, the swing arm 230 can be configured to guide movement of the cartridge 106 between the first receptacle 210 and the second receptacle 212. In in some aspects, at least a portion of the actuator 220 (e.g., the calipers 222) can be coupled to the swing arm 230 so that the actuator can travel with the cartridge 106 as the cartridge moves between the first receptacle 210 and the second receptacle 212. In further alternative aspects, the calipers 222 can couple directly to the cartridge 106, and the swing arm 230 can move the cartridge with the calipers coupled to the cartridge.

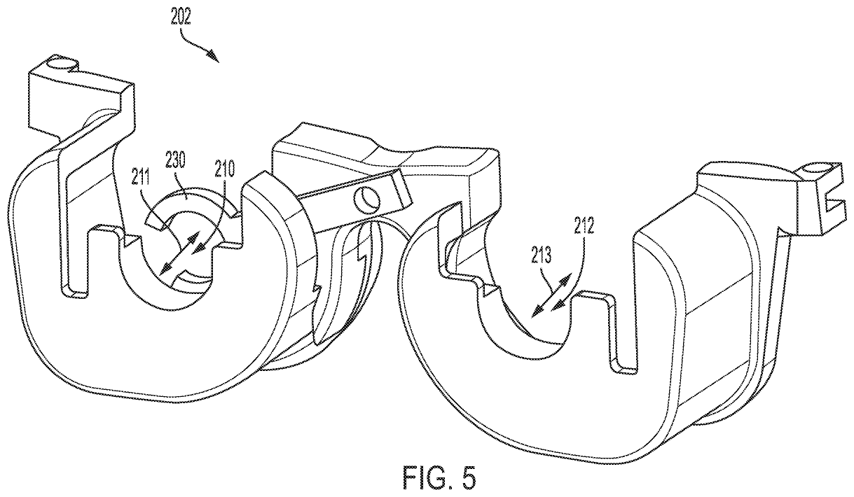

To use the eye drop dispensing apparatus 200, a user or caretaker can insert a cartridge 106 into the receptacle of the frame 202. For example, optionally, the user can position the cartridge 106 within the swing arm 230 and move the swing arm to the first position (FIG. 5) in which the swing arm aligns the cartridge is received within the first receptacle, and the outlet of the cartridge is aligned (e.g., coaxial) with the central axis 211 of the first receptacle 210. The user or a caretaker can align the apparatus 200 so that the outlet 108 of the cartridge 106 is directly above the user's eye. For example, the frame 202 can be placed over the face of the user with the user's head tilted back. The user or caretaker can actuate the actuator 220 (e.g., pivot the lever toward the handle, thereby retracting the cable and drawing the calipers 222 toward the central axis) to dispense the predetermined quantity of solution from the apparatus.

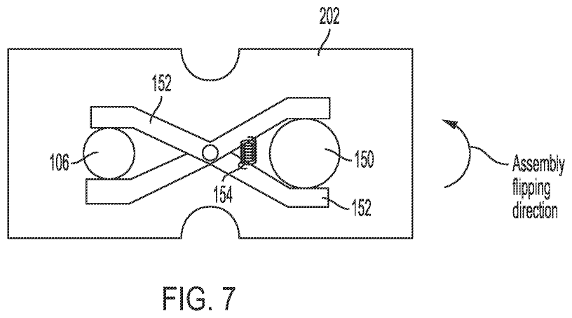

Referring to FIG. 7, in further aspects, the actuator 120 or actuator 220 can comprise a bladder 150. Optionally, the user can actuate a hand pump or electronic pump to cause the bladder 150 to expand in some aspects, expansion of the bladder 150 can expand outwardly to actuate pivot ably coupled lever arms 152 to compress the cartridge 106. Optionally, the lever arms 152 can comprise gripping elements (e.g., rubber gripping elements that can serve as engagement elements) that engage an outer surface of the cartridge 106. In some optional aspects, a spring or other biasing element 154 can bias the lever arms 152 in respective pivotal directions away from the cartridge 106 to return the lever arms to a starting position (e.g., not compressing the cartridge) after compressing the cartridge. Optionally, the frame 202 can be reversible (e.g., rotated 180 degrees in a flipping direction) so that the assembly as shown in FIG. 7 can selectively be positioned over each eye.

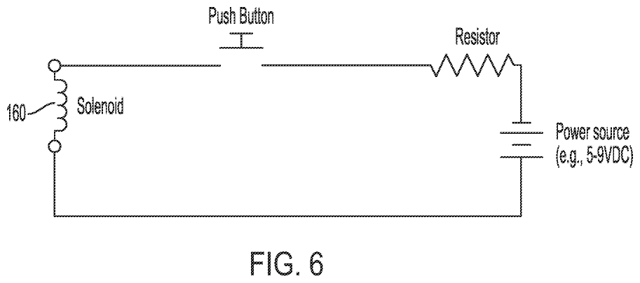

In still further aspects, referring to FIG. 6, the actuator 120 or actuator 220 can comprise a solenoid 160. The solenoid can be actuated to bias against the cartridge 106. The solenoid can optionally be actuated via wired connection (as shown) or wirelessly (e.g., via a wireless remote or other wireless controller, not shown).

Although several embodiments of the invention have been disclosed in the foregoing specification and the following appendices, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed herein, and that many modifications and other embodiments are intended to be included within the scope of the appended claims. Moreover, although specific terms are employed herein, as well as in the claims Which follow, they are used only in a generic and descriptive sense, and not. for the purposes of limiting the described invention, nor the claims which follow.

What is claimed is:

1. An apparatus comprising:
a housing that defines at least a first receptacle that is configured to receive and support a cartridge having a solution therein, the first receptacle having a central axis; and
an actuator that is coupled to the housing and movable between a first position and a second position, wherein the actuator comprises an engagement element,
wherein in the first position, the engagement element of the actuator is radially spaced from the central axis of the first receptacle by a first distance, and
wherein in the second position, the engagement element of the actuator is positioned within the first receptacle and radially spaced from the central axis of the first receptacle by a second distance that is less than the first distance.

2. The apparatus of claim 1, wherein the housing defines an alignment feature that is at least partially receivable into an eye socket of a user so that when the housing is positioned above an eye of the user and the alignment feature is received within the eye socket of the user, the alignment feature is configured to align the central axis of the first receptacle with the eye of the user.

3. The apparatus of claim 1, wherein the actuator comprises a rack and a pinion, wherein the rack defines the engagement element of the actuator.

4. The apparatus of claim 3, wherein the pinion comprises a lever.

5. The apparatus of claim 1, further comprising a stop that is configured to inhibit the actuator from moving, beyond the second position.

6. The apparatus of claim 1, wherein the housing comprises a frame that defines at least one alignment feature, wherein the at least one alignment feature is configured to engage a face of a user to align the frame in a predetermined orientation with respect to the farce of the user, wherein the first receptacle is configured to align a cartridge containing solution therein with respect to a first eye of the user, and wherein the housing further defines a second receptacle that is configured to align the cartridge containing solution therein with a second eye of the user.

7. The apparatus of claim 6, wherein the frame defines the first receptacle and the second receptacle.

8. The apparatus of claim 6, wherein the actuator comprises a pair of calipers and a cable, wherein the pair of calipers are configured to close upon retraction of the cable.

9. The apparatus of claim 6, wherein the housing further comprises a swing arm that is configured to receive the cartridge and move with the cartridge between the first receptacle and the second receptacle.

10. The apparatus of claim 1, further comprising the cartridge, wherein the cartridge contains an eye drop solution.

11. A method comprising:
positioning a cartridge within a first receptacle of a housing of an apparatus, the cartridge containing a solution, the first receptacle having a central axis, the apparatus further comprising an actuator that is coupled to the housing and movable between a first position and a second position, wherein the actuator comprises an engagement element, wherein in the first position, the engagement element of the actuator is radially spaced from the central axis of the first receptacle by a first distance; and
moving the actuator from the first position to the second position, Wherein in the second position, the engagement element of the actuator is positioned within the first receptacle and radially spaced from the central axis of the first receptacle by a second distance that is less than the first distance,
wherein in the second position, the engagement element of the actuator compresses the cartridge to dispense a quantity of the solution within the cartridge.

12. The method of claim 11, wherein the housing defines an alignment feature, the method further comprising:
with the alignment feature of the housing positioned at least partially within an eye socket of an eye of aa user, positioning the housing above the eye of the user, wherein the alignment feature aligns the central axis of the first receptacle with the eye of the user.

13. The method of claim 11, wherein the actuator comprises a rack and a pinion, wherein the rack defines the engagement element of the actuator.

14. The method of claim 13, wherein the pinion comprises a lever.

15. The method of claim 11, wherein the apparatus further comprises a stop that inhibits the actuator from moving beyond the second position.

16. The method of claim 11, wherein the housing comprises a frame that defines at least one alignment feature, the method further comprising:
engaging a face of a user with the at least one alignment feature to align the frame in a predetermined orientation with respect to the face of the user,
wherein the first receptacle is configured to align the cartridge with respect to a first eye of the user, and wherein the housing further defines a second receptacle that is configured to align the cartridge containing solution therein with a second eye of the user.

17. The method of claim 16, wherein the frame defines the first receptacle and the second receptacle.

18. The method of claim 16, wherein the actuator comprises a pair of calipers and a cable, wherein the pair of calipers are configured to close upon retraction of the cable.

19. The method of claim 16, wherein the housing further comprises a swing arm, the method further comprising:
positioning the cartridge within the swing arm; and
moving the swing arm from a first rotational position to a second rotational position, wherein in the first rotational position, the cartridge is positioned within the first receptacle, and wherein in the second rotational position, the cartridge is positioned within the second receptacle.

20. The method of claim 11, wherein the cartridge contains an eye drop solution.
